(19) United States
(12) Patent Application Publication
(10) Pub. No.: US 2009/0138043 A1
(43) Pub. Date: May 28, 2009

(54) THREADED ACCESS CANNULA AND METHODS OF USING THE SAME

(75) Inventor: Andrew Kohm, San Mateo, CA (US)

Correspondence Address: COOLEY GODWARD KRONISH LLP;ATTN: PATENT GROUP, Suite 1100, 777 - 6th Street, NW, WASHINGTON, DC, 20001, US

(73) Assignee: Medtronic Spine LLC, Sunnyvale, CA (US)

(21) Appl. No.: 11/946,199

(22) Filed: Nov. 28, 2007

(51) Int. Cl. A61B 17/70; A61B 17/86; A61B 17/56

(52) U.S. Cl. 606/246; 606/301; 606/93

(57) ABSTRACT

Medical devices and methods for accessing a biological body are disclosed herein. In one embodiment, a method includes inserting a cannula at least partially into a vertebra such that a threaded portion of the cannula secures the cannula to a portion of a cortical bone of the vertebra and forms a channel within the cortical bone. A medical device is inserted at least partially through the cannula such that a distal end portion of the medical device is disposed within a portion of a cancellous bone of the vertebra. A medical procedure is performed within the cancellous bone using the medical device and then the cannula and the medical device are removed from the vertebra, leaving a channel in the vertebra having at least partially threaded interior walls. A bone screw that has threads configured to matingly engage the threaded channel is then inserted into the threaded channel.

### BACKGROUND

The invention relates generally to medical devices and procedures, including, for example, a medical device for providing percutaneous access to a biological body.

Known medical devices are configured to access percutaneously a vertebra, an intervertebral disc, or other area of a body, to perform a variety of different medical procedures. Some known medical devices are configured to remove tissue from, for example, an interior of a vertebra or intervertebral disc. Other known medical devices are configured to inject bone cement or other types of prosthesis into a biological body. A cannula or other type of access device is typically used to provide a pathway for other medical devices to be inserted into a biological body to perform such procedures. Often, multiple tool changes may be needed during a procedure making it desirable to maintain the position of the cannula within the biological body.

Thus, a need exists for an apparatus and method for accessing a biological body, such as a vertebra or intervertebral disc, and maintaining a position of the access device during one or more medical procedures being performed within the biological body.

### SUMMARY OF THE INVENTION

Medical devices and methods for accessing a biological body are disclosed herein. In one embodiment, an apparatus includes a cannula having an anchoring portion (e.g., threaded outer surface, tapered wings, etc.) configured to secure the position of the cannula after the distal portion of the cannula has been inserted into a tissue. In one embodiment, a method includes inserting a cannula at least partially into a vertebra such that a threaded portion of the cannula secures the cannula to a portion of a cortical bone of the vertebra. A medical device is inserted at least partially through the cannula such that a distal end portion of the medical device is disposed within a portion of a cancellous bone of the vertebra. A medical procedure is performed within the cancellous bone using the medical device and then the cannula and medical device are removed from the vertebra, leaving a threaded channel in the vertebra (i.e., a channel having at least partially threaded interior walls). A bone screw is then inserted into the threaded channel. The bone screw has threads configured to matingly engage the threaded channel.

### BRIEF DESCRIPTION OF THE DRAWINGS

FIG. 1 is a schematic illustration of a medical device according to an embodiment of the invention.

FIG. 2 is a side view of a medical device according to an embodiment of the invention.

FIG. 3 is a side view of a medical device according to another embodiment of the invention.

FIG. 4 is a side view of a portion of a medical device shown disposed partially within a portion of a bone.

FIG. 5 is a side view of a portion of the medical device of FIG. 4 shown further disposed within the portion of the bone.

FIG. 6 is a top view of a medical device according to an embodiment of the invention shown partially inserted into a vertebra.

FIG. 7 is a top view of the medical device of FIG. 6 shown partially inserted into the vertebra, and another medical device shown disposed through the medical device and partially within the vertebra.

FIG. 8 is a side view shown partially in cross-section of a medical device according to another embodiment of the invention.

FIG. 9 is a side view of a portion of a threaded portion of a medical device according to an embodiment of the invention.

FIG. 10 is a side view of a portion of a threaded portion of a medical device according to an embodiment of the invention shown in a collapsed configuration.

FIG. 11 is a side view of the portion of the threaded portion of the medical device of FIG. 10 shown in an expanded configuration.

FIG. 12 is a cross-sectional view of the medical device of FIG. 10 taken along line 12-12 in FIG. 10.

FIG. 13 is a cross-sectional view of the medical device of FIG. 11 taken along line 13-13 in FIG. 11.

FIG. 14 is a side view of a portion of the medical device of FIGS. 10-13 shown partially inserted into hard tissue and soft tissue.

FIG. 15 is a top view of a medical device according to an embodiment of the invention shown partially inserted into a vertebra.

FIG. 16 is a top view of the vertebra of FIG. 15 after the medical device has been removed from the vertebra.

FIG. 17 is a top view of the vertebra of FIGS. 15 and 16 with a bone screw shown partially inserted into a channel formed in the vertebra.

FIG. 18 is a side view of an intervertebral disc, the adjacent vertebra and a portion of a medical device shown partially disposed within the intervertebral disc.

FIG. 19 is a side view of a stylet according to an embodiment of the invention.

FIGS. 20-23 are each a flowchart illustrating a method according to different embodiments of the invention.

### DETAILED DESCRIPTION

The devices and methods described herein are configured for percutaneous insertion into an interior area of a patient's body, such as within a hard tissue area (e.g., bone structure) or soft tissue area (e.g., intervertebral disc) of a patient and to provide access to such areas. For example, a medical device disclosed herein can provide access to an interior of a vertebra or other area of a spinal column. Some embodiments described herein include a cannula having a threaded portion that can be threadably secured to a biological body, such as a vertebra. A medical device can be inserted through the cannula and into the biological body and used to perform a procedure. The threaded securement of the cannula to the biological body provides stability to the cannula and enables the cannula to maintain its position relative to the biological body.

In some embodiments, a portion of a cannula can be forcibly inserted at least partially into a biological body and then another portion of the cannula can be threadably turned or screwed into the biological body. The combination of forcibly inserting and rotatingly screwing the cannula into the biological body can reduce insertion time and the effort required to insert the cannula into the biological body.

In some embodiments, an apparatus can be secured to hard tissue areas (e.g., cortical bone) of a vertebra and provide access to soft tissue areas (e.g. cancellous bone) of the vertebra. With the cannula secured to the vertebra, an expandable medical device, such as, for example, a device with an inflatable balloon tamp, can be inserted into the cancellous bone of the vertebra and expanded such that a cavity is produced within the cancellous bone. The expandable medical device can then be removed and another device can be used to inject bone cement into the vertebra. For example, another device can be inserted through the lumen of the cannula and used to inject bone cement into the vertebra. In some embodiments, bone cement can be injected directly through a lumen of the cannula and into the vertebra. For example, a bone cement delivery device (e.g., an injection needle) can be coupled to a proximal end of the cannula and used to inject bone cement into the vertebra via the lumen of the cannula.

It is noted that, as used in this written description and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, the catheter end inserted inside a patient's body would be the distal end of the catheter, while the catheter end outside a patient's body would be the proximal end of the catheter.

In one embodiment, a method includes inserting a cannula at least partially into a vertebra such that a thread forming portion of the cannula secures the cannula to a portion of cortical bone of the vertebra. A medical device is inserted at least partially through the cannula such that a distal end portion of the medical device is disposed within a portion of a cancellous bone of the vertebra. A medical procedure is performed within the cancellous bone using the medical device and then the cannula and medical device are removed from the vertebra, leaving a channel within the cortical bone having at least partially threaded interior walls. A bone screw is then inserted into the threaded channel. The bone screw has threads configured to matingly engage the threaded channel.

In another embodiment, a method includes applying a non-rotary force to a cannula in a distal direction such that an anchoring portion of the cannula having multiple collapsible threads is at least partially inserted into a biological body and at least one thread is moved from a collapsed configuration to an expanded configuration within the biological body. The cannula defines a lumen that is configured to receive a medical device therethrough. In some embodiments, the cannula can be moved proximally to further engage at least a portion of the anchoring portion of the cannula with the biological body.

In another embodiment, a method includes applying a non-rotary distal force to a cannula such that a distal end portion of the cannula is at least partially inserted into an annulus of an intervertebral disc. After applying the non-rotary distal force, the cannula is rotated such that a threaded portion of the cannula is at least partially threaded into the intervertebral disc to secure the cannula to the annulus of the intervertebral disc. A distal end portion of a medical device is inserted through a lumen of the cannula and at least a distal portion of the medical device is placed into a nucleus of the intervertebral disc.

In another embodiment, an apparatus includes a cannula that defines a lumen that extends between a proximal end portion and a distal end portion of the cannula and has an anchoring portion on an outer surface. The anchoring portion includes multiple collapsible anchor members each having a collapsed configuration for insertion into a biological body and being movable to an expanded configuration to secure the cannula to the biological body.

In another embodiment, an apparatus includes a cannula that has an elongate portion defining a lumen extending between a proximal end portion and a distal end portion of the cannula. The cannula has a threaded portion that includes multiple threads and is at a non-zero distance from a distal end of the cannula. The threaded portion is configured to be inserted into a biological body when a force in a distal direction is applied to the cannula and configured to be secured to the biological body when a non-rotary force in a proximal direction is applied to the cannula.

In another embodiment, an apparatus includes a cannula that defines a lumen that extends between a proximal end portion and a distal end portion of the cannula. The cannula includes a threaded portion disposed on an outer surface of the cannula. An elongate member (e.g., a stylet) is configured to be movably disposed within the lumen of the cannula. The elongate member has a threaded portion disposed on an outer surface of a distal end portion of the elongate member and a distal tip configured to penetrate biological tissue. The cannula and the elongate member are collectively configured to be inserted into a biological body when a portion of the elongate member is disposed within the lumen of the cannula and a distal end of the cannula is disposed between the distal tip of the elongate member and a proximal end of the elongate member.

In another embodiment, a method includes inserting a medical device at least partially into a biological body. The medical device includes a cannula and a stylet disposed within a lumen of the cannula. After inserting the medical device, a distal end of the stylet is moved outside a distal end of the cannula. The stylet is rotated such that a threaded portion of the stylet penetrates the biological body. The cannula is rotated such that a threaded portion of the cannula threadably secures the cannula to the biological body.

In another embodiment, a kit is provided that includes a cannula having a thread-forming portion on an outer wall of the cannula. The cannula is configured to be inserted into a bone such that when the cannula is removed from the bone, a channel is formed in the bone having at least partially threaded interior walls. The kit also includes at least one bone screw having a threaded portion configured to matingly engage the threaded interior walls of the channel formed by the cannula. In some embodiments, the kit also includes a medical device configured to be inserted at least partially through a lumen of the cannula when the cannula is inserted at least partially into a biological body. The medical device can be used to perform a medical procedure within the biological body.

The term “tissue” is used here to mean an aggregation of similarly specialized cells that are united in the performance of a particular function. For example, a tissue can be a soft tissue area (e.g., a muscle), a hard tissue area (e.g., a bone structure), a vertebral body, an intervertebral disc, a tumor, etc. The terms “body” and “biological body” are also referred to herein to have a similar meaning as the term tissue.

The term “cannula” is used here to mean a component of the apparatus having one or more passageways configured to receive a device or other component. For example, a cannula can be substantially tubular. A cannula can be a variety of different shapes and size, such as having a round, square, rectangular, triangular, elliptical, or octagonal inner and/or outer perimeter.

The term “tapped” is used herein to modify terms such as hole or channel. For example, a tapped hole or a tapped channel is a hole or channel that has been formed with grooves or internal threads such that the hole or channel receives a screw or threaded member and the grooves or internal threads of the hole or channel matingly receive or complimentarily fit with the external threads of the screw or threaded member. The term “tap” is used herein to mean the act, step or process of forming a tapped hole or a tapped channel, or can refer to a tool configured to form internal screw threads.

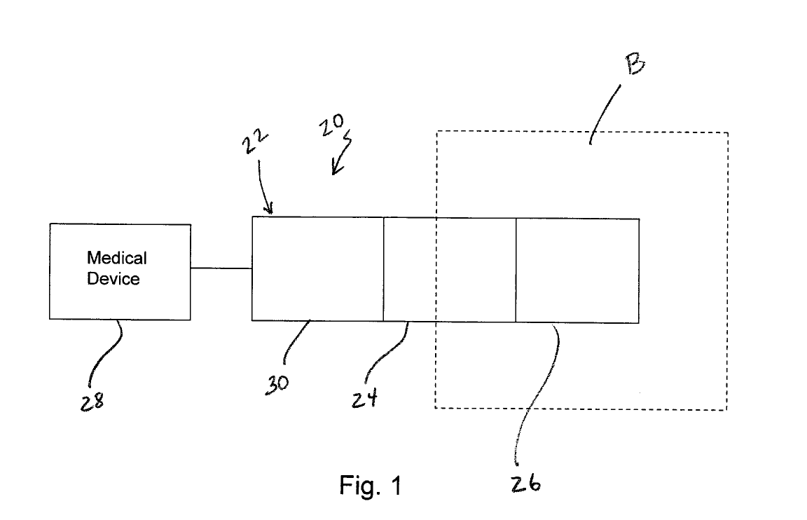

FIG. 1 is a schematic illustration of a medical device according to an embodiment of the invention. A medical device 20 (also referred to herein as cannula) includes an elongate member 22 that defines a lumen (not shown in FIG. 1) between a proximal end portion and a distal end portion of the elongate member 22. The lumen can be configured to receive another medical device 28 therethrough. For example, in some embodiments, a stylet can be inserted through the lumen and used to penetrate a biological body when the stylet and the cannula are inserted into the biological body. Other medical devices 28 can be inserted through the lumen of the elongate member 22 and used to perform a medical procedure within a biological body as described in more detail below.

The cannula 20 also includes a threaded portion 24 on an outer surface of the distal end portion of the elongate member 22 and an unthreaded portion 30 on an outer surface proximally of the threaded portion 24. The cannula 20 can also optionally include an unthreaded portion 26 disposed on an outer surface distally of the threaded portion 24. The threaded portion 24 can be any of a variety of different lengths along a length of the elongate member 22, and have any of a variety of different configurations. For example, the threaded portion 24 can include threads having various lengths and angles relative to the outer surface of the elongate member 22. The threaded portion 24 can also include collapsible threads that can collapse during insertion into a biological body and then can be expanded or unfolded to secure the cannula 20 to the biological body. Each of these features is described in more detail below with reference to specific embodiments.

In one use, the cannula 22 can be used to tap a hole in a biological body for later insertion of a screw. For example, the cannula 20 can be used to tap a hole in a pedicle of a vertebra and a pedicle screw can later be threaded into the tapped hole. In some embodiments, a stylet is first inserted into a lumen of the elongate member 22 such that a distal end of the stylet is disposed outside the lumen at a distal end of the cannula 20. The distal end of the stylet can be configured to penetrate percutaneously a biological body as the cannula 20 is being inserted. For example, a distal end portion of the stylet can be beveled, angled, pointed, arrow shaped or any other configuration that allows the stylet to penetrate a biological body. In some embodiments, a stylet can include threads on an outer surface of a distal end portion of the stylet. In some embodiments, a stylet can include a lumen extending between a proximal portion and a distal portion of the stylet. The lumen of the stylet can be slidably moved over a guidewire that can guide the positioning of the stylet and/or cannula during insertion.

The cannula 20 can also be percutaneously inserted into a biological body of a patient, such as a vertebral body, and used to provide access to the biological body. For example the cannula can be threadably inserted into a biological body such that the threaded portion 24 of the cannula 20 threadably secures the cannula 20 to the biological body. The threaded securement of the cannula 20 to the biological body provides stability to the cannula 20 and maintains the position of the cannula 20 relative to the biological body. In some embodiments, if the cannula 20 includes an unthreaded portion 26 distally of the threaded portion 24, the cannula 20 can first be forcibly inserted at least partially into the biological body. For example, the distal force can be exerted on a proximal end portion of the cannula 20 such that the unthreaded portion 26 is driven or pushed into the biological body. The cannula 20 can then be turned or threaded into the biological body using the threaded portion 24. Such an embodiment can reduce insertion time and the amount of rotating or turning effort required to insert the cannula 20 into the biological body.

With the cannula 20 secured to the biological body, a variety of different medical procedures can be performed using the cannula 20 to access the biological body. For example, the cannula 20 can be secured to a portion of cortical bone of a vertebra and provide access to cancellous bone of the vertebra. A medical device having an expandable member can be inserted into the cancellous bone and expanded to produce a cavity within the cancellous bone. Such a medical device can include, for example, a mechanically-actuated expandable member, or an expandable balloon, such as those typically used in cavity-producing procedures. After the cavity has been produced, the medical device can be removed from the cannula 20 and another device, such as a bone-cement delivery device, can be inserted through the cannula 20 or coupled to a proximal end portion of the cannula 20, and used to inject bone cement into the cavity. In some embodiments, an implantable prosthesis can be inserted into the cavity.

These are just examples of the types of procedures that can be performed using the cannula 20. For example, procedures to disrupt or otherwise cut tissue can be performed using the cannula 20 to gain access to a desired tissue site. Tissue removal procedures can also be performed using the cannula 20. Thus, although the embodiments illustrated focus on the use of the cannula 20 in a vertebra, it should be understood that the cannula 20 can be used in other biological bodies, such as other areas within a spinal column, or other hard bone or soft tissue areas within a body.

Having described above various general examples, several examples of specific embodiments are now described. These embodiments are only examples, and many other configurations of a medical device 20 are contemplated.

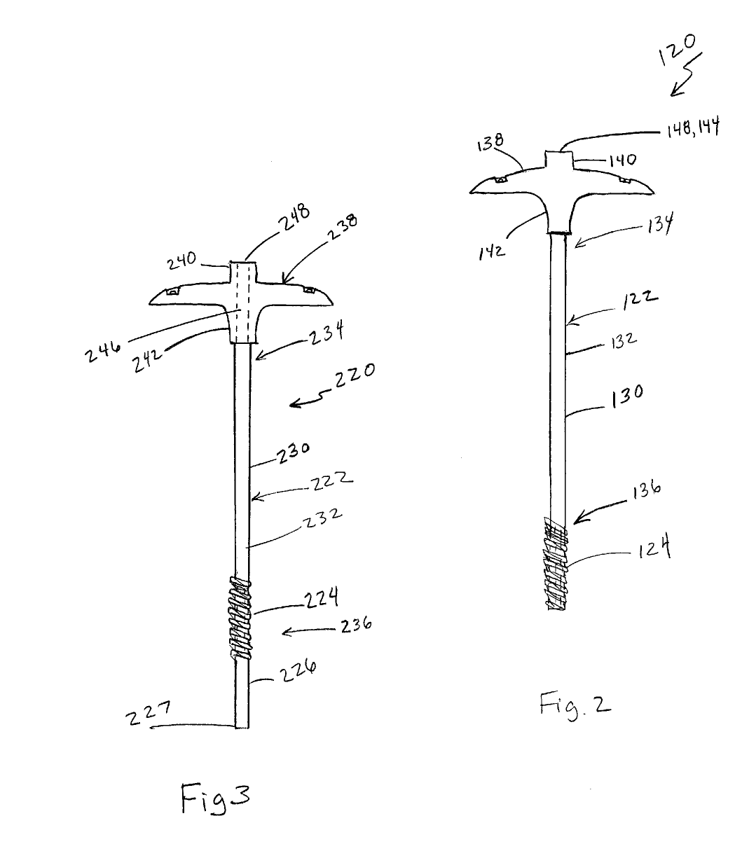

FIG. 2 illustrates a cannula 120 according to an embodiment of the invention. The cannula 120 includes an elongate member 122 that defines a lumen (not shown) that extends between a proximal end portion 134 and a distal end portion 136 of the elongate member 122. In this embodiment, the elongate member 122 includes a threaded portion 124 disposed on the distal end portion 136, and an unthreaded portion 130 proximal of the threaded portion 124. The threaded portion 124 can be used to threadably secure the cannula 120 to a biological body as described above and as described in more detail below.

The cannula 120 also includes a handle 138 coupled to the elongate member 122. The elongate member 122 can extend through a lumen (not shown) in the handle 138 and terminate at a proximal end portion 140 of the handle 138. The elongate member 122 defines an opening 144 at a proximal end of the elongate member 122 that is in communication with the lumen 132. The handle 138 defines a an opening 148 in communication with the opening 144 of the elongate member 122. Thus, various medical devices (not shown in FIG. 2) can be inserted through the opening 148 (of the handle 138) and the opening 144 (of the elongate member 122) and at least partially within the lumen of the elongate member 122, as described above.

In some embodiments, the elongate member 122 does not extend through the handle 138 as described above, but instead is coupled to a distal end portion 142 of the handle 138. In such an embodiment, the handle 138 can define a lumen that extends between the proximal end portion 140 and the distal end portion 142 of the handle 138 and that communicates with the lumen 132 of the elongate member 122. A medical device can then be inserted through the lumen of the handle and through the lumen of the elongate member 122.

FIG. 3 illustrates a cannula according to another embodiment. A cannula 220 includes an elongate member 222 coupled to a handle 238. The elongate member 222 defines a lumen (not shown) that extends between a proximal end portion 234 and a distal end portion 236 of the elongate member 222. The handle 238 also defines a lumen 246 in communication with the lumen of the elongate member 222 and an opening 248 in communication with the lumen 246 of the handle 238. In this embodiment, the elongate member 222 includes a threaded portion 224 disposed on the distal end portion 236 at a spaced or non-zero distance from a distal end 227 of the elongate member 222. For example, the threaded portion 224 (e.g., a distal end of the threaded portion) can be at about 10 mm to about 100 mm from the distal end 227 of the elongate member 222. The elongate member 222 includes an unthreaded portion 230 disposed proximally of the threaded portion 224, and an unthreaded portion 226 disposed distally of the threaded portion 224.

As with the previous embodiments, the threaded portion 224 can be used to threadably secure the cannula 220 to a biological body, however, in this embodiment, the unthreaded portion 226 can be used to first forcibly move or insert a portion of the cannula 220 into a biological body. For example a non-rotary distal force can be exerted on the cannula 220 to move the unthreaded portion 226 into a biological body. The cannula 222 can then be turned or rotated to engage the threads of the threaded portion 224 with the biological body and threadably secure the cannula 220 thereto.

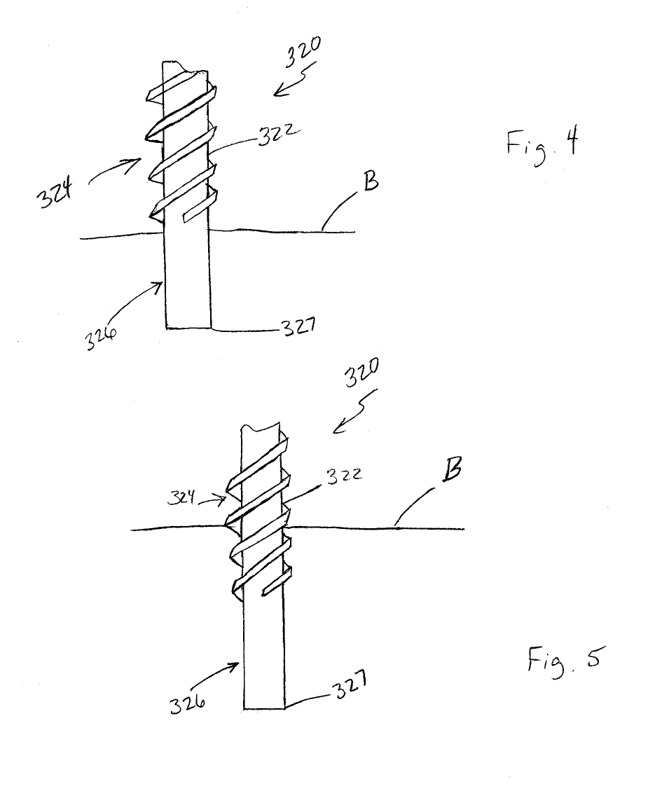

FIGS. 4 and 5 illustrate an example of the insertion of a distal end portion of a cannula configured similar to the cannula 220. As shown in FIG. 4, a cannula 320 includes an elongate member 222 having an unthreaded portion 326 and a threaded portion 324 disposed at a non-zero distance from a distal end 327 of the elongate member 322, and proximally of the unthreaded portion 326. FIG. 4 illustrates the unthreaded portion 326 of the elongate member 322 after being forcibly moved into a biological body B. For example, a non-rotary force in a distal direction can be applied to the cannula 320 to forcibly move the cannula 320 distally. After inserting the unthreaded portion 326, the cannula 320 can be turned or rotated to threadably engage the biological body B with the threaded portion 324, as shown in FIG. 5. Thus, the initial insertion time of the cannula 320 can be reduced by delaying the start of the rotation of the threaded portion 324 into the biological body B due to the initial application of force to the cannula.

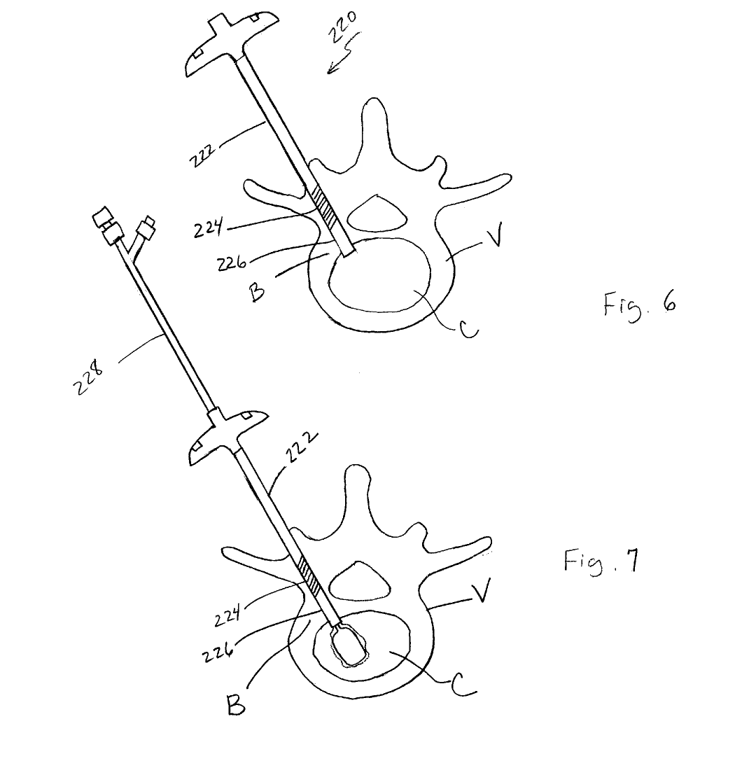

FIGS. 6 and 7 illustrate the cannula 220 inserted into a vertebra V and used as an access pathway for another medical device to perform a medical procedure within the vertebra V. As shown in FIG. 6, the cannula 220 is inserted into a vertebra V in the same manner as described above with reference to FIGS. 4 and 5, such that the unthreaded portion 226 is disposed within cancellous bone C, and the threaded portion 224 is at least partially threadably engaged with a hard tissue area (e.g. cortical bone B) of the vertebra V. As shown in FIG. 7, a medical device 228 is inserted through the lumen (shown in FIG. 3) of the elongate member 222 of the cannula 220, and a distal end portion 250 of the medical device 228 is disposed within the cancellous bone C. The distal end portion 250 includes a balloon shown in an expanded configuration to form a cavity within the cancellous bone C. The threaded portion 224 of the cannula 220 stabilizes the cannula 220 during such a procedure. Although not shown, additional medical procedures can be performed in the vertebra V while maintaining the position of the cannula 220 relative to the vertebra V. For example, the medical device 228 can be removed after producing the cavity in the cancellous bone C and a second device (not shown) such as a bone cement delivery device can then be inserted through the cannula 220 and into the vertebra.

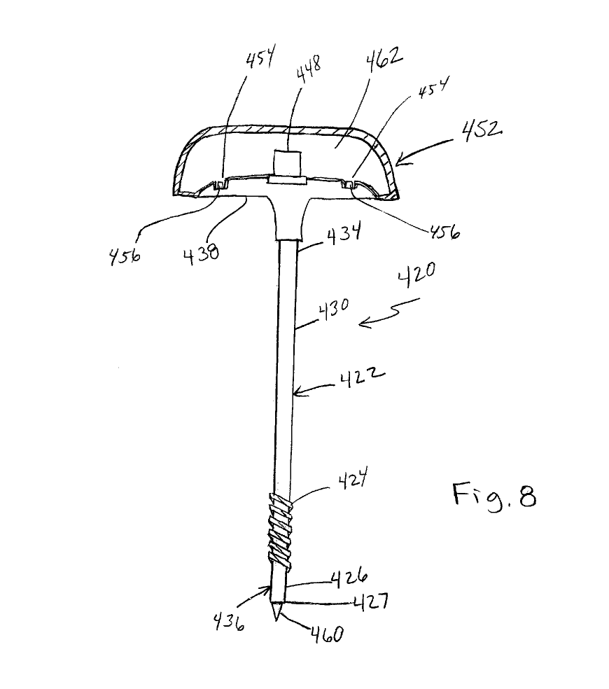

FIG. 8 illustrates an embodiment of a cannula configured to matingly couple to a stylet. A cannula 420 includes an elongate member 422 coupled to a handle 438. The elongate member 422 defines a lumen (not shown in FIG. 8) extending between a proximal end portion 434 and a distal end portion 436 of the elongate member 422. As with previous embodiments, the handle 438 also defines a lumen (not shown in FIG. 8) in communication with the lumen (not shown in FIG. 8) of the elongate member 422. In this embodiment, the elongate member 422 includes an unthreaded portion 426 disposed at the proximal end portion 436, and a threaded portion 424 disposed proximally of the unthreaded portion 426 and at a non-zero distance from a distal end 427 of the elongate member 422. The elongate member 422 also includes an unthreaded portion 430 disposed proximally of the threaded portion 424.

In this embodiment, the handle 438 is configured to matingly couple to a handle 462 (shown in cross-section in FIG. 8) of a stylet device 452. For example, the handle 438 includes protrusions 456 configured to engage corresponding coupling portions 454 of the stylet device 452. The stylet device 452 includes an elongate portion (not shown) configured to be movably disposed within the lumen of the elongate member 422. The stylet device 452 also includes a distal end 460 configured to penetrate biological tissue (e.g., angled, beveled, pointed) and configured to be disposed outside the distal end of the elongate member 422, as shown in FIG. 8.

With the stylet device 452 coupled to the cannula 420, the cannula 420 and stylet device 452 can collectively be inserted into a biological body. For example, a non-rotary distal force can be applied to the cannula 422 and stylet device 452 to forcibly insert the unthreaded portion 426 of the elongate member 422 into the biological body. The cannula 420 can then be turned or rotated to threadably advance the threaded portion 424 of the elongate member 422 into the biological body. As these actions are performed, the distal end 460 of the stylet device 452 can penetrate the biological body defining a pathway into the biological body.

In some embodiments, the stylet can also define a lumen extending between a proximal end and a distal end of the stylet. In such an embodiment, the stylet can be moved over a guidewire to assist in the insertion process. For example, a guidewire (not shown) can be inserted into a pedicle of a vertebra such that a distal end of the guidewire is positioned within the pedicle. The stylet (and cannula coupled to the stylet) can then be slid over the guidewire (e.g., guidewire disposed within the lumen of the stylet) and into the pedicle of the vertebra. This procedure can be used to guide the proper positioning of the cannula within the vertebra. The distal tip of the stylet can then be used to penetrate the tissue of the vertebra and position the cannula at a desired location relative to and partially within the vertebra.

As with the previous embodiments, the threaded portion 424 can threadably secure the cannula 420 to the biological body such that other medical procedures can be performed within the biological body. For example, after the cannula 422 is secured to the biological body, the stylet device 452 can be detached from the handle 438 and removed from the cannula 420. Another medical device (not shown in FIG. 8) can then be inserted through an opening 448 in the handle 438, through the lumens of the handle 438 and elongate member 422, and into the biological body. The medical device can then be used to perform a medical procedure within the biological body.

The threaded portion of the cannulas described herein can include a variety of different thread sizes, shapes, and/or configurations, including thread-forming features (rather than continuous threads). FIG. 9 is an enlarged view of a distal end portion of a threaded portion of a cannula illustrating an example embodiment of threads. As shown in FIG. 9, a cannula 520 includes an elongate member 522 having a threaded portion 524. The threaded portion 524 includes multiple threads 564 (only two threads 564 are illustrated in FIG. 9). In this example embodiment, the threads 564 are generally shallow and steep relative to the outer surface 566 of the elongate member 522, which allows the threads 564 to be easily inserted into a biological body (e.g., when the cannula is hammered or tapped in a distal direction), but resist pullout from the biological body. For example, the threads 564 define a length L, and an angle θ relative to the outer surface 566 of the elongate member 522. The angle θ can be, for example, an acute angle. In some embodiments, the angle is between, for example, 20 and 85 degrees. The length L can be, for example, between 0.25 mm and 2 mm.

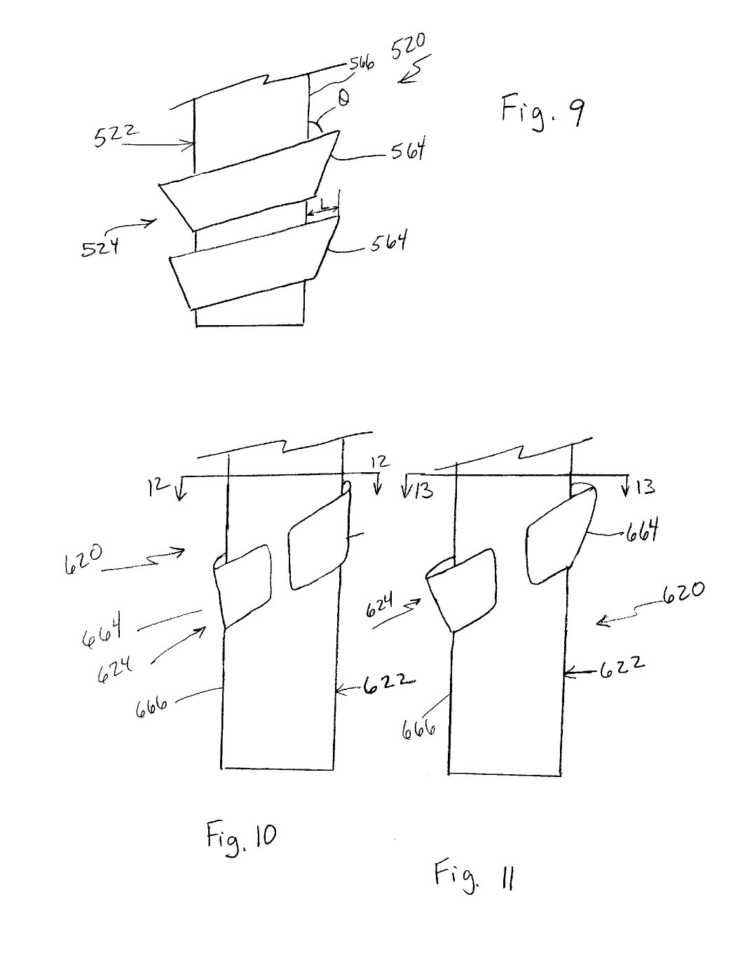

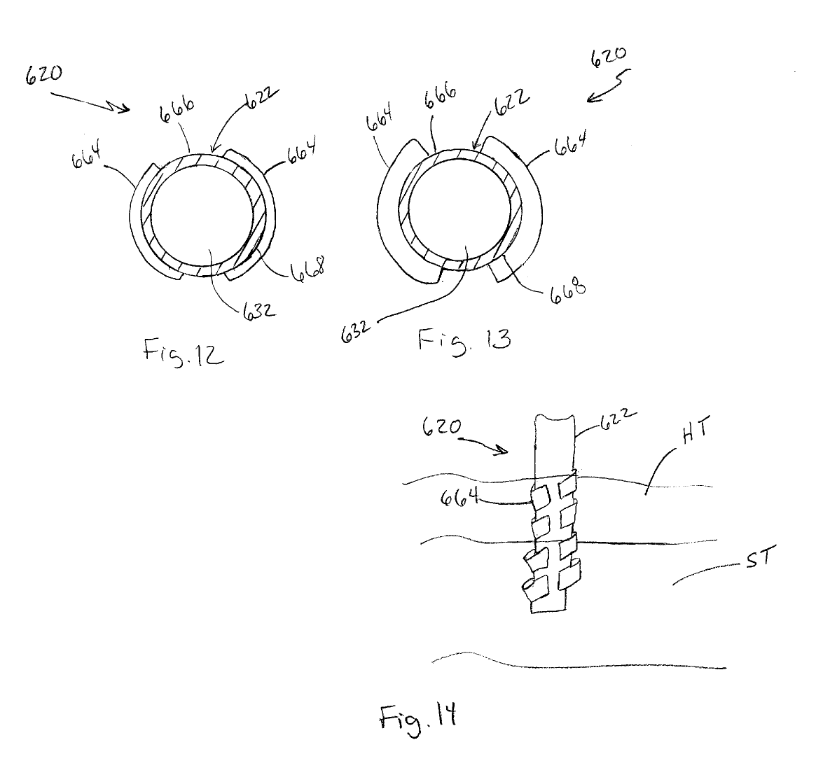

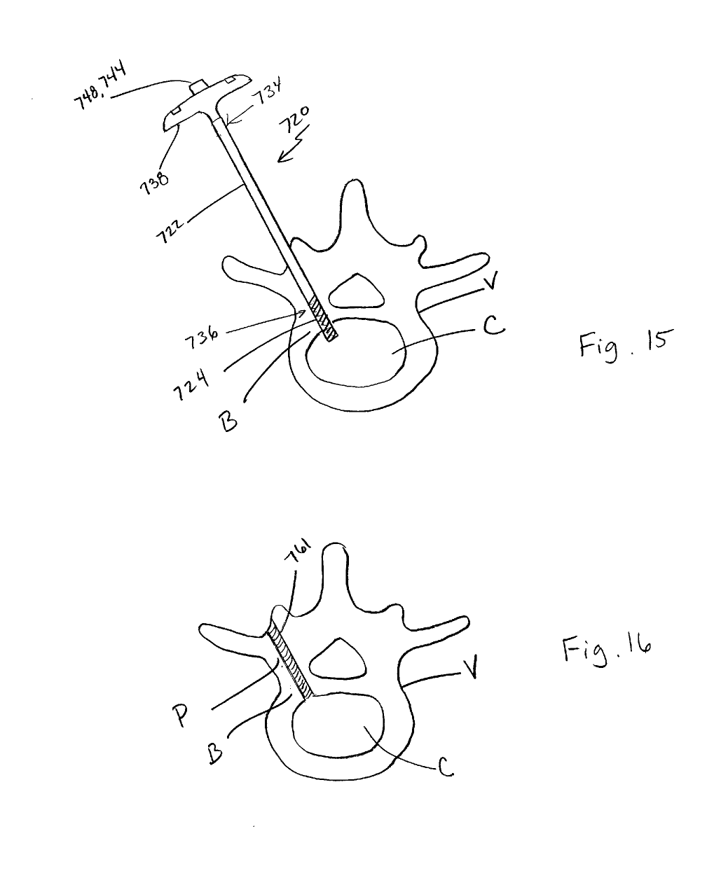

FIGS. 10-13 illustrate another example embodiment of threads for a threaded portion of a cannula. FIGS. 10 and 11 are each an enlarged view of a distal end portion of a cannula 620 having an elongate member 622 including a threaded portion 624. The elongate member 622 also includes an outer surface 666 and a lumen 632 (shown in FIGS. 12 and 13). In this embodiment, the threaded portion 624 includes threads 664 that are split into two-parts each disposed on opposite sides of the elongate member 622. Although only one such thread 664 is illustrated, the threaded portion 624 of the cannula 620 can include multiple threads 664.

The threaded portion 624 has a collapsed configuration, as shown in FIGS. 10 and 12, and an expanded configuration, as shown in FIGS. 11 and 13. For example, the threads 664 can be formed to collapse and expand as described below. The elongate member 622 and threads 664 can be formed with a deformable material, such as a deformable plastic. In some embodiments, the threads 664 can form a hinge at a base 668 of the threads 664 where the threads 664 meet the surface 666 as shown in FIGS. 12 and 13. The threads 664 can also be formed with a deformable shape-memory material and biased into the expanded configuration. The threads 664 can be collapsed when a lateral force is applied to the threads 664 (e.g., in a direction toward the surface 666). The collapsibility of the threads 664 allow the cannula 620 to be inserted into a biological body when a distal force is applied to the cannula 620. As the cannula 620 is being inserted, the threads 664 will fold or collapse closer to the surface 666 as shown in FIGS. 10 and 12. After insertion of the cannula 620 through, for example, hard or semi-hard tissue, and into softer tissue, the threads 664 can assume their biased or expanded configuration.

FIG. 14 illustrates the insertion of a distal end portion of the cannula 620 through hard tissue HT and into soft tissue ST. As shown in FIG. 14, some of the threads 664 are shown collapsed within the hard tissue HT, and some are shown biased into the expanded configuration as they enter into the soft tissue ST. After the threaded portion 624 of the cannula is fully in the soft tissue, the cannula 620 can then be moved proximally and rotated such that at least some of the threads 664 threadably engage the hard tissue HT. In other words, the threads 664 can be threaded or screwed into tissue (e.g., hard tissue) of a biological body from an interior region (e.g., having softer tissue) in a direction toward an exterior of the biological body (e.g., inside-out).

In some embodiments, after insertion of the threaded portion 624 into the softer tissue region of a biological body, the threads 664 can remain in a folded or collapsed. configuration. In such an embodiment, to move the threads 664 to an expanded configuration, the cannula 620 can be moved proximally such that the threads 664 engage the tissue of the biological body and are moved to the expanded configuration.

In one example use, the cannula 620 can be inserted into a vertebra by first applying a distal force to the cannula 620 such that the distal end portion of the cannula 620 is inserted through the cortical bone of the vertebra and into the cancellous bone. The threads 664 will fold or collapse during insertion as described above. After being inserted into the softer cancellous bone, the threads 664 will be biased back to their expanded or unfolded configuration. The threads 664, while in the expanded configuration, can then be screwed or threaded into the cortical bone of the vertebra by rotating and moving the cannula 620 proximally. This action will threadably secure the cannula 620 to the cortical bone of the vertebra and the cannula 620 can then be used to access the vertebra in the same manner as described for previous embodiments. The cannula 620 can be removed from the biological body by again rotating the cannula 620 and moving the cannula 620 proximally (e.g., unscrewing the cannula 620).

FIGS. 15-17 illustrate the use of another embodiment of a cannula. A cannula 720 includes an elongate member 722 coupled to a handle 738. The elongate member 722 defines a lumen (not shown) that extends between a proximal end portion 734 and a distal end portion 736 of the elongate member 722. The handle 738 also defines a lumen (not shown) in communication with the lumen of the elongate member 722 and an opening 748 in communication with the lumen of the handle 738. In this embodiment, the elongate member 722 includes a threaded portion 724 disposed on the distal end portion 736 of the elongate member 722 and an unthreaded portion 730 disposed proximally of the threaded portion 724.

As with the previous embodiments, the threaded portion 724 can be used to threadably secure the cannula 720 to a biological body, in the same manner as previously described. For example, the cannula 720 can be rotated while a distal force is applied to the cannula 720 to advance the cannula 720 into a biological body, such as a vertebra V, as shown in FIG. 15. The threaded portion 724 can engage a portion of the cortical body B of the vertebra and secure the cannula 720 with a distal end 727 of the cannula 720 disposed within a portion of the cancellous bone C of the vertebra V. As described above for previous embodiments, various medical devices can be inserted through a lumen (not shown) of the cannula and used to perform a medical procedure within the vertebra V. For example, similar to as shown in FIG. 7 for cannula 220, an expandable device (e.g., a balloon) can be inserted through the cannula 720, and positioned within a distal end portion within the cancellous bone C of the vertebra V. The expandable device can then be expanded to form a cavity within the cancellous bone C. Other procedures, such as tissue removal procedures, bone cement injection, etc. can alternatively or also be performed.

After the desired medical procedure is performed, the cannula 720 can then be removed leaving a threaded channel 761 as shown in FIG. 16. The threaded channel 761 can be formed or tapped during insertion of the cannula 720 into the vertebra V. For example, as the cannula 720 is advanced into the vertebra V, the threaded portion 724 cuts internal threads on interior walls of the channel 761 being formed by the cannula 720. The threaded or tapped channel 761 can be used to secure a bone screw 763, such as a pedicle screw, to for example a pedicle P of the vertebra V, as shown in FIG. 17. The bone screw 763 can be configured with threads configured to matingly engage the threaded interior walls of the channel 761. A bone screw can “matingly” engage the threads of the threaded channel even if the bone screw threads do not exactly match the dimension/pitch/profile/etc. of the threaded channel, so long as the bone screw can be at least partially screwed into the threaded channel (e.g., the use of mismatched threads to effectively lock the bone screw in the threaded channel). A fusion device (not shown) can then be secured to one or more vertebra after performing a medical procedure within the cancellous bone C of the vertebra V. Using the cannula 720 to tap the channel 761 can thus, eliminate the need to use a separate device to tap a hole for insertion of a bone screw.

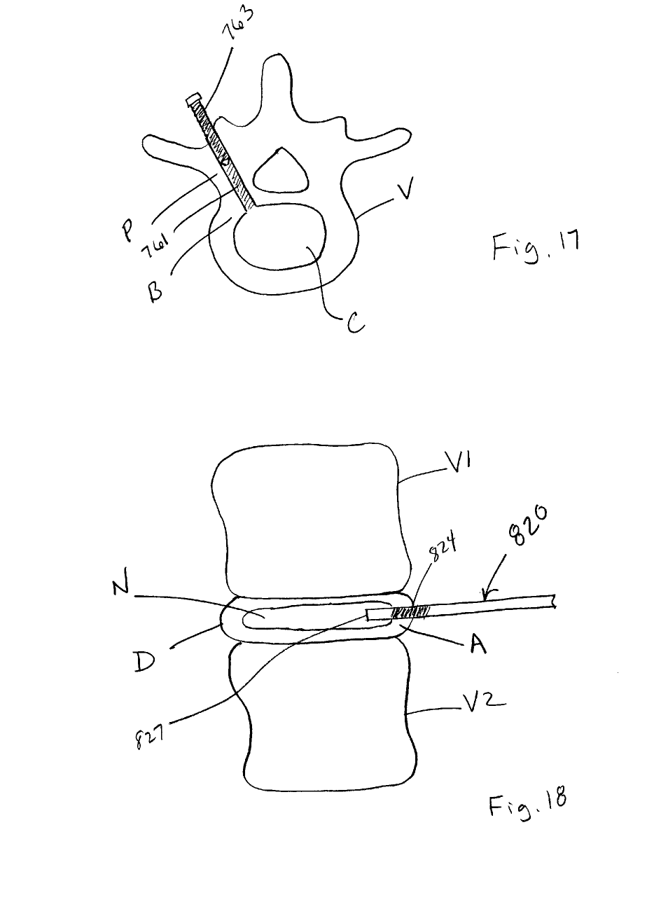

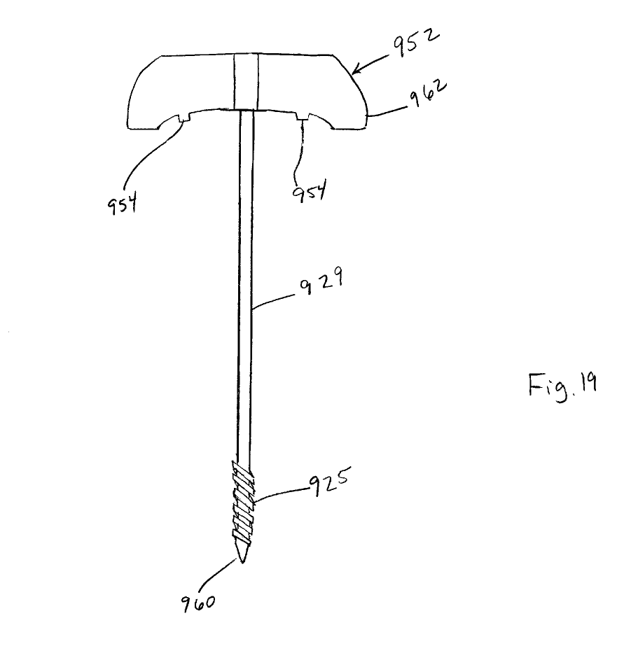

FIG. 18 illustrates the use of a cannula 820 in an example of a procedure within an intervertebral disc. The cannula 820 can be configured in the same manner as any of the previously described embodiments, and include an anchoring portion 824 (e.g., threads). Only a portion of the cannula 820 is shown in FIG. 18. The cannula 820 can be inserted through an annulus wall A of an intervertebral disc D in the same manner as described above or previous embodiments. A distal end 827 of the cannula 820 can be disposed within the nucleus N of the intervertebral disc D. The anchoring portion 824 can be used to secure and stabilize the cannula 820 to the intervertebral disc D and provide access to the interior of the intervertebral disc D. A medical device can be inserted through the lumen of the cannula 820 and used to perform a medical procedure, such as nucleus disruption, nucleus removal, insertion of an implant within the nucleus, or distraction of adjacent vertebra V1 and/or V2, with, for example, an expandable device.

In addition to a cannula including an anchoring portion as described herein, a stylet can also include an anchoring portion. FIG. 19 illustrates a stylet 952 having a threaded portion 925 disposed on a distal end portion of the stylet 920. The stylet 952 also includes an unthreaded portion 929 coupled to a handle 962 and a distal tip portion 962. The threaded portion 925 can be adjacent to the distal tip portion 962 or be at a non-zero distance from the distal tip portion 962. The stylet 952 can include a lumen (not shown) extending between a proximal end portion and a distal end portion of the stylet 952, or the stylet 952 can be at least partially solid (e.g., no lumen). The threaded portion 925 can be used to help advance the stylet 952 through a biological body. The threaded portion 925 of the stylet 952 can also be used to disrupt tissue within the biological body. For example, the stylet 952 can be inserted into an interior region of a biological body, such as within cancellous bone of a vertebra or a nucleus of an intervertebral disc, and then rotated such that the threaded portion 925 disrupts tissue within the biological body.

The stylet 952 can be used in conjunction with a cannula that includes an anchoring portion (e.g., threaded portion) or with a cannula that does not include an anchoring portion. The stylet 952 can be configured to be releasably secured to a cannula as described above for the embodiment of FIG. 8. For example, the stylet 952 can include coupling portions 954 that matingly engage coupling portions on a handle of a cannula.

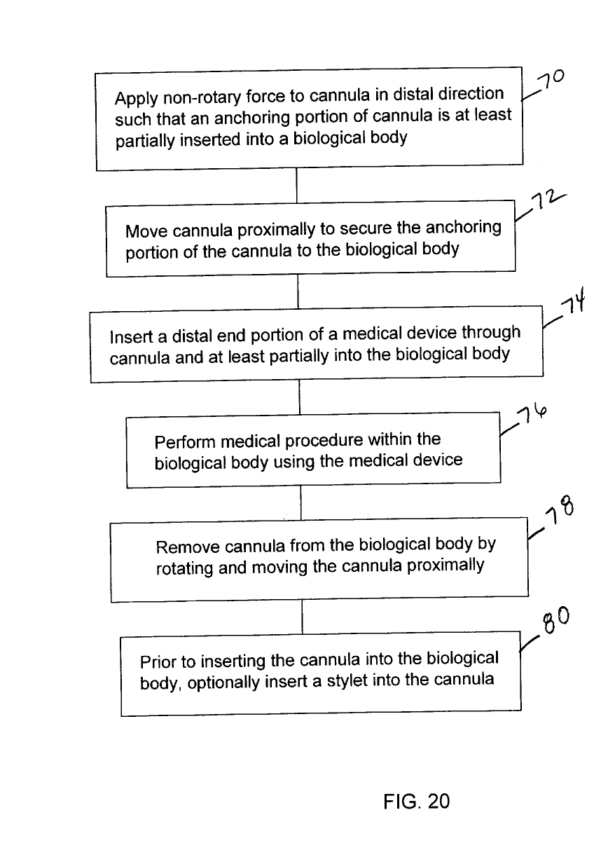

FIG. 20 is a flowchart illustrating a method of using a cannula according to an embodiment of the invention. A method includes at 70 applying a non-rotary force to a cannula in a distal direction such that an anchoring portion of the cannula is at least partially inserted into a biological body. The biological body can be, for example, a vertebra or an intervertebral disc. The anchoring portion of the cannula can include, for example, multiple collapsible anchor members at least some of which are moved from a collapsed configuration to an expanded configuration when the cannula is inserted into the biological body. The cannula can also define a lumen configured to receive a medical device therethrough. At 72, the cannula can be moved proximally to secure the anchoring portion of the cannula to the biological body. In some embodiments, the cannula is moved proximally and rotated such that the anchoring portion (e.g., threaded portion) engages the biological body and secures the cannula to the biological body. In some embodiments the anchor members can be collapsible threads as described herein and the cannula is rotated while being moved such that the threads threadably secure the cannula to the biological body.

With the cannula secured to the biological body, at 74, a distal end portion of a medical device can optionally be inserted through the lumen of the cannula and at least partially into the biological body. The medical device can optionally have an expandable portion. At 76, a medical procedure can be performed within the biological body using the medical device. For example, an expandable portion of the medical device can be expanded such that a cavity is formed within the biological body. At 78, the cannula can be removed from the biological body by rotating and moving the cannula proximally. For example, the cannula can be rotated to threadably removed from the biological body. At 80, prior to inserting the cannula into the biological body, a stylet can be optionally inserted into the lumen of the cannula such that a distal end of the stylet is disposed outside of a distal end of the cannula. The distal end of the stylet can be configured to penetrate the biological body.

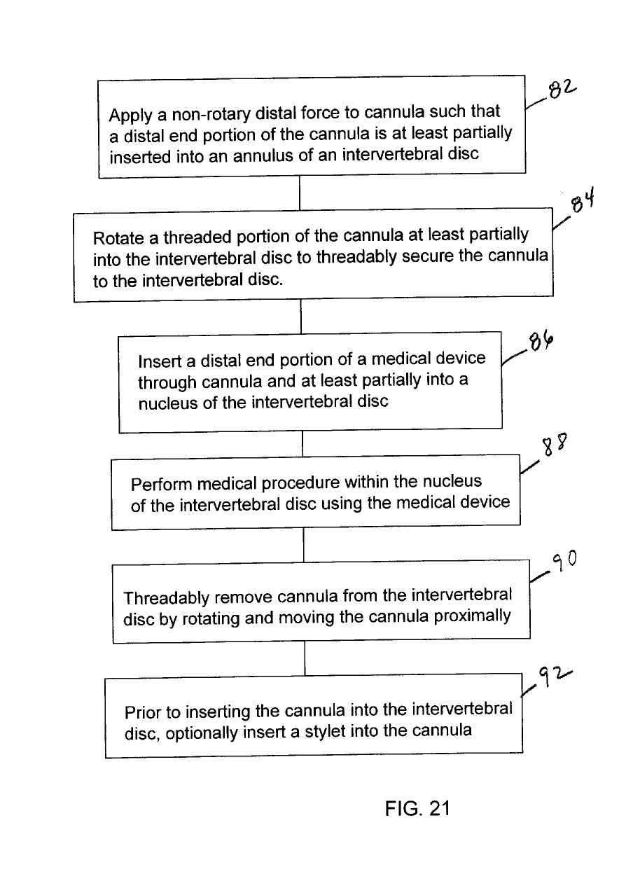

FIG. 21 is a flowchart illustrating another method of using a cannula according to an embodiment of the invention. At 82, a distal force is applied to a cannula such that a distal end portion of the cannula is at least partially inserted into an annulus of an intervertebral disc. The distal end portion of the cannula can be smooth (e.g., unthreaded). At 84, after applying the distal force to the cannula, a threaded portion of the cannula can be turned or rotated at least partially into the annulus of the intervertebral disc to threadably secure the cannula to the intervertebral disc. For example, a distal force can be applied while rotating the cannula to screw the cannula into the intervertebral disc.

At 86, a distal end portion of a medical device can optionally be inserted through a lumen of the cannula and at least partially into a nucleus of the intervertebral disc. At 88, a medical procedure can be performed within the nucleus of the intervertebral disc using the medical device. For example, an expandable device can be expanded within the nucleus such that an endplate of an adjacent vertebra is moved. In another example, a medical procedure can be performed to remove nucleus material from within the intervertebral disc. At 90, the cannula can be threadably removed from the intervertebral disc by rotating and moving the cannula proximally. At 92, prior to inserting the cannula into the biological body at 82, a stylet can be optionally inserted into the lumen of the cannula such that a distal end of the stylet is disposed outside of a distal end of the cannula. The distal end of the stylet can be configured to penetrate the intervertebral disc.

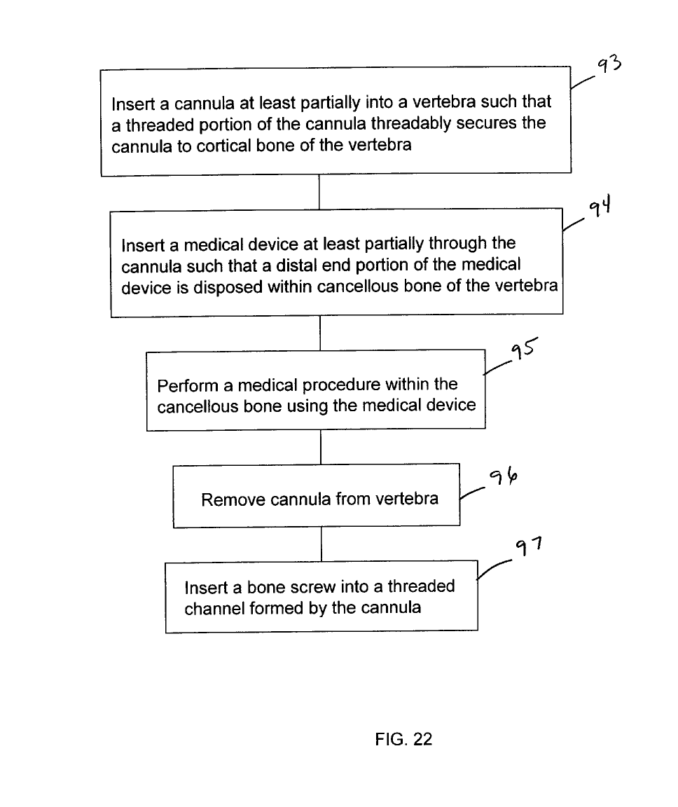

FIG. 22 is a flowchart illustrating an example of a method of using a cannula to form a tapped channel. A method includes at 93, inserting a cannula at least partially into a vertebra such that a threaded portion of the cannula threadably secures the cannula to cortical bone of the vertebra and forms a channel within the cortical bone that has at least partially threaded interior walls. At 94, a medical device is inserted at least partially through the cannula such that a distal end portion of the medical device is disposed within cancellous bone of the vertebra. At 95, a medical procedure is performed within the cancellous bone using the medical device. For example, a medical procedure can include a kyphoplasty procedure, a vertebroplasty procedure, and/or a procedure to remove cancellous bone from the interior of the vertebra. At 96, the cannula is removed from the vertebra. At 97, a bone screw, such as a pedicle screw can be inserted into the threaded channel formed by the cannula. For example, a bone screw can have threads configured to matingly engage the threaded channel formed by the cannula in the vertebra.

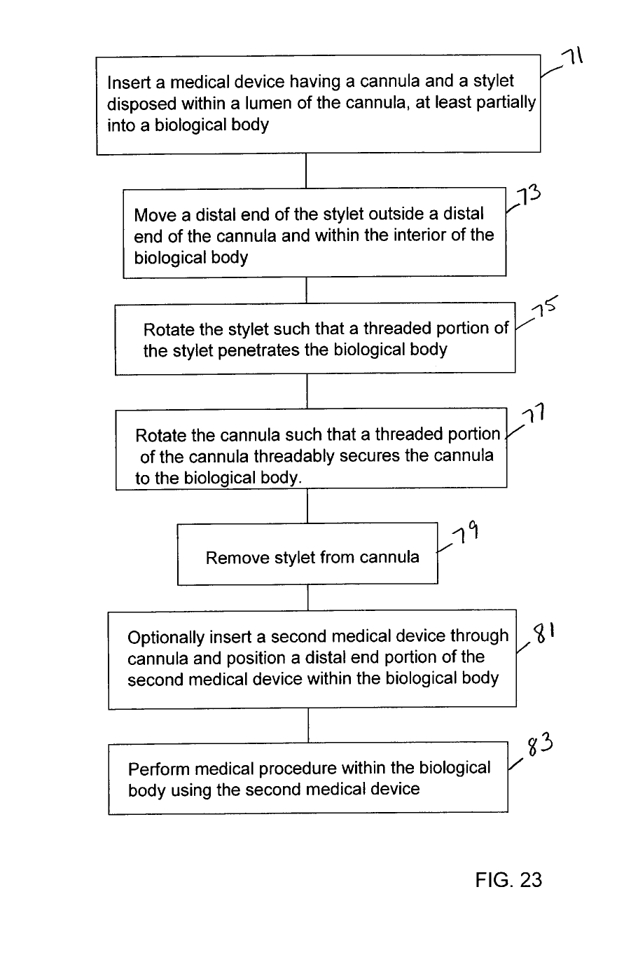

FIG. 23 is a flowchart of another example of a method of using medical device including a cannula and a stylet. A method includes at 71, inserting a medical device having a cannula and a stylet disposed within a lumen of the cannula, at least partially into a biological body. The biological body can be, fore example, a vertebra or an intervertebral disc. At 73, after inserting the medical device, a distal end of the stylet is moved outside a distal end of the cannula and within the interior of the biological body. At 75, the stylet is rotated such that a threaded portion of the stylet penetrates the biological body. At 77, the cannula is rotated such that a threaded portion of the cannula threadably secures the cannula to the biological body.

At 79, after the rotating the cannula, the stylet can be removed from the cannula. At 81, a second medical device can optionally be inserted through a lumen of the cannula and a distal end portion of the second medical device is positioned within an interior of the biological body. At 83, a medical procedure can be performed within the biological body using the second medical device. For example, a distal end portion of the second medical device can be expanded within the interior of the biological body such that a cavity is formed within the biological body. In another example, the biological body is an intervertebral disc and a distal end portion of the second medical device is expanded within the nucleus of the intervertebral disc such that an endplate of an adjacent vertebra is moved.

The various components described herein can also be included in a kit. For example, a kit can include a threaded cannula according to any of the embodiments described herein (e.g., cannula 20, 120, 220, 320, 420, 520, 620, 720, 820) and a medical device(s) configured to be inserted at least partially through a lumen of the cannula. The medical device can be, for example, an expandable device (e.g., medical device 228) configured to be inserted into, for example, a vertebra, and then expanded. The medical device can be, for example, a balloon bone tamp. The kit can also optionally include a stylet (e.g., stylet 452, 952) configured to be inserted through a lumen of the cannula, as described herein.

In another example, a kit can include a cannula having a threaded or thread-forming portion on an outer wall configured to form a threaded channel in a bone (as described herein), and at least one bone screw that can be matingly received within the threaded channel. The bone screw (or screws) can be, for example, a pedicle screw. Such a kit can also optionally include a medical device configured to be inserted at least partially through a lumen of the cannula and into a biological body as described above. The kit can also include a stylet as described above and/or a fusion rod configured to be secured to a bone structure(s) with the bone screw(s). For example, a kit can include a cannula 720 and a bone screw 763 shown in FIGS. 15 and 17, respectively, along with any other optional devices/tools to be used with cannula 720 (e.g., stylet(s), bone tamp(s), bone cement injector(s), and/or any other complementary devices/tools).

The various components of the medical devices described herein can be constructed with any suitable material used for such a medical device. For example, the elongate member and/or handle for any embodiment can be formed with various biocompatible metals, such as stainless steel, titanium, titanium alloy, surgical steel, metal alloys, or suitable biocompatible plastic materials, such as various polymers, polyetheretherketone (PEEK), carbon fiber, ultra-high molecular weight (UHMW) polyethylene, etc., or combinations of various materials thereof.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having a combination or sub-combination of any of the features and/or components from any of the embodiments discussed above. For example, the cannula can include threaded and unthreaded portions at various locations along its length, and the length of the threaded and unthreaded portions can vary. Although not shown, the distal end of the elongate member for any embodiment can be configured to penetrate a tissue (e.g., include a sharp distal end, such as beveled, pointed, angled, etc.).

In addition, any of the embodiments of a medical device can include other types of threads not specifically shown or described. For example, the threads can vary in size, shape, angle, length, quantity, etc. The cannulas described herein can be configured to provide access to a variety of different medical devices, to perform a variety of different medical procedures.

Further, the various components of a medical device as described herein can have a variety of different shapes and or size not specifically illustrated. For example, the handle can have various different configurations not specifically shown. A cannula can include more than one lumen and the lumen(s) can be a variety of different shapes and sizes. For example, a cross-section of a lumen can be, circular, oval, square, rectangular, triangular, oblong, etc.

What is claimed is:

1. A method, comprising:
inserting a cannula at least partially into a vertebra such that a thread-forming portion of the cannula secures the cannula to a portion of cortical bone of the vertebra;
inserting a medical device at least partially through the cannula such that a distal end portion of the medical device is disposed within a portion of cancellous bone of the vertebra;
performing a medical procedure within the portion of cancellous bone using the medical device;
removing the cannula and the medical device from the vertebra and leaving a channel within the cortical bone, the channel having at least partially threaded interior walls; and
threadably inserting a bone screw into the threaded channel after the removing, the bone screw having threads configured to matingly engage the threaded channel.

2. The method of claim 1, wherein the medical device is a first medical device, the first medical device includes an expansion member and the performing includes forming a cavity within the cancellous bone of the vertebra with the expansion member, the method further comprising:
inserting a second medical device at least partially through the cannula; and
injecting a bone cement into the cavity formed by the expansion member.

3. The method of claim 1, further comprising:
securing a fixation rod to the vertebra via the bone screw.

4. The method of claim 1, wherein the channel is formed at least partially within a pedicle of the vertebra.

5. The method of claim 1, wherein the channel is formed at least partially within a pedicle of the vertebra, the threadably inserting includes threadably inserting a pedicle screw into the channel.

6. A method, comprising:
applying a non-rotary force to a cannula in a distal direction such that an anchoring portion of the cannula having a plurality of collapsible anchor members is at least partially inserted into a biological body and at least one anchor member from the plurality of anchor members is moved from a collapsed configuration to an expanded configuration, the cannula defining a lumen configured to receive a medical device therethrough.

7. The method of claim 6, further comprising:
moving the cannula proximally to secure at least a portion of the anchoring portion of the cannula to the biological body.

8. The method of claim 6, wherein the plurality of collapsible anchor members include a plurality of collapsible threads disposed on an exterior surface of the cannula, the method further comprising:
rotating the cannula such that at least one anchor member threadably secures the cannula to the biological body.

9. The method of claim 6, wherein the biological body is a vertebra, the applying includes inserting at least a portion of the anchoring portion of the cannula through a portion of cortical bone of the vertebra and into a portion of cancellous bone of the vertebra.

10. The method of claim 6, wherein the biological body is an intervertebral disc, the plurality of collapsible anchor members include a plurality of collapsible threads, the applying includes inserting at least a portion of the anchoring portion of the cannula through a portion of an annulus of the intervertebral disc and into a portion of a nucleus of the intervertebral disc, the method further comprising:
moving the cannula proximally and rotating the cannula such that the portion of the anchoring portion of the cannula is threaded into the portion of annulus of the intervertebral disc.

11. The method of claim 6, wherein the biological body is a vertebra, the method further comprising:
moving the cannula proximally to secure at least a portion of the anchoring portion of the cannula to the vertebra;
after the moving, inserting a distal end portion of an expandable medical device through the lumen of the cannula and into a portion of cancellous bone of the vertebra;
expanding an expandable portion of the medical device such that a cavity is formed within the portion of cancellous bone of the vertebra; and
after the expanding, injecting a bone cement into the cavity.

12. A method, comprising:
applying a non-rotary distal force to a cannula such that a distal end portion of the cannula is at least partially inserted into a portion of an annulus of an intervertebral disc;
after the applying, rotating the cannula such that a threaded portion of the cannula is at least partially advanced into the intervertebral disc to threadably secure the cannula to the portion of the annulus of the intervertebral disc; and
inserting a distal end portion of a medical device through a lumen of the cannula; and
placing at least a distal portion of the medical device into a portion of a nucleus of the intervertebral disc.

13. The method of claim 12, further comprising:
after the inserting, performing a medical procedure within the nucleus of the intervertebral disc using the medical device.

14. The method of claim 12, wherein the medical device includes an expandable portion, the method further comprising:
after the inserting, expanding the expandable portion of the medical device such that a cavity is formed within of the nucleus of the intervertebral disc.

15. The method of claim 12, wherein the medical device includes an expandable portion, the method further comprising:
after the inserting, expanding the expandable portion of the medical device such that an endplate of a vertebra adjacent to the intervertebral disc is moved.

16. The method of claim 12, further comprising:
threadably removing the cannula from the intervertebral disc.

17. The method of claim 12, further comprising:
prior to the inserting, inserting a stylet into the lumen of the cannula such that a distal end of the stylet is disposed outside of a distal end of the cannula, the distal end of the stylet configured to penetrate the intervertebral disc.

18. An apparatus, comprising:
a cannula defining a lumen extending between a proximal end portion and a distal end portion of the cannula, the cannula having an anchoring portion on an outer surface of the cannula,
the anchoring portion including a plurality of collapsible anchor members each having a collapsed configuration for insertion into a biological body and being movable to an expanded configuration to secure the cannula to the biological body.

19. The apparatus of claim 18, wherein the plurality of anchor members are a plurality of threads configured to threadably secure the cannula to the biological body.

20. The apparatus of claim 18, wherein a distal end of the anchoring portion is disposed at a non-zero distance from a distal end of the cannula,

21. The apparatus of claim 18, further comprising:
a stylet removably disposable within the lumen of the cannula such that a distal end of the stylet is disposed outside of the lumen of the cannula, the stylet including a handle configured to be removably coupled to a handle of the cannula.

22. The apparatus of claim 18, further comprising:
a stylet removably disposable within the lumen of the cannula such that a distal end of the stylet is disposed outside of the lumen of the cannula, the distal end of the stylet configured to penetrate the biological body.

23. An apparatus, comprising:
a cannula having an elongate portion defining a lumen extending between a proximal end portion and a distal end portion of the cannula, and the cannula having a threaded portion at a non-zero distance from a distal end of the cannula,
the threaded portion including a plurality of threads, the threaded portion configured to be inserted into a biological body when a force in a distal direction is applied to the cannula and configured to be secured to the biological body when a non-rotary force in a proximal direction is applied to the cannula.

24. The apparatus of claim 23, further comprising:
a stylet removably disposable within the lumen of the elongate portion such that a distal end of the stylet is disposed outside of the lumen of the elongate portion, the stylet including a handle configured to be removably coupled to a handle of the cannula.

25. The apparatus of claim 23, further comprising:
a stylet removably disposable within the lumen of the elongate portion such that a distal end of the stylet is disposed outside of the lumen of the elongate portion, the distal end of the stylet configured to penetrate the biological body.

26. The apparatus of claim 23, wherein the threaded portion of the cannula includes a plurality of collapsible threads, each of the threads from the plurality of collapsible threads having a collapsed configuration for insertion into the biological body and are movable to an expanded configuration to threadably secure the cannula to the biological body.

27. An apparatus, comprising:
a cannula defining a lumen extending between a proximal end portion and a distal end portion of the cannula, the cannula including a threaded portion disposed on an outer surface of the cannula; and
an elongate member configured to be movably disposed within the lumen of the cannula, the elongate member having a threaded portion disposed on an outer surface of a distal end portion of the elongate member and a distal tip configured to penetrate biological tissue,
the cannula and the elongate member collectively configured to be inserted into a biological body when a portion of the elongate member is disposed within the lumen of the cannula and a distal end of the cannula is disposed between the distal tip of the elongate member and a proximal end of the elongate member.

28. The apparatus of claim 27, wherein the threaded portion of the cannula includes a plurality of threads, each thread from the plurality of threads defining an angle relative to an outer surface of the elongate portion of the cannula and having a length,
the angle and length of each thread from the plurality of threads being configured such that the cannula can be inserted into a biological body when a non-rotary force in a distal direction is applied to the cannula and such that the cannula can be secured to the biological body when the cannula is rotated proximally.

29. The apparatus of claim 27, wherein the angle is an acute angle.

30. The apparatus of claim 27, wherein the angle is between about 20 degrees and 85 degrees, the length of the threads is between about 0.25 mm and 2 mm.

31. The apparatus of claim 27, wherein the elongate member includes a handle, the cannula includes a handle, the handle of the elongate member configured to be matingly and releasably coupled to the handle of the cannula.

32. A method, comprising:
inserting a medical device at least partially into a biological body, the medical device including a cannula and a stylet disposed within a lumen of the cannula;
after the inserting, moving a distal end of the stylet outside a distal end of the cannula;
rotating the stylet such that a threaded portion of the stylet penetrates the biological body; and
rotating the cannula such that a threaded portion of the cannula threadably secures the cannula to the biological body.

33. The method of claim 32, further comprising:
after the rotating the cannula, removing the stylet from the cannula.

34. The method of claim 32, wherein the medical device is a first medical device, the method further comprising:
after the rotating the cannula, removing the stylet from the cannula;
after the removing, inserting a second medical device through a lumen of the cannula; and
positioning a distal end portion of the second medical device within an interior of the biological body.

35. The method of claim 32, wherein the medical device is a first medical device, the method further comprising:
after the rotating the cannula, removing the stylet from the cannula;
after the removing, inserting a second medical device through a lumen of the cannula;
positioning a distal end portion of the second medical device within an interior of the biological body; and
expanding the distal end portion of the second medical device within the interior of the biological body such that a cavity is formed within the biological body.

36. The method of claim 32, wherein the biological body is a vertebra, the medical device is a first medical device, the method further comprising:
after the rotating the cannula, removing the stylet from the cannula;
after the removing, inserting a second medical device through a lumen of the cannula;
positioning a distal end portion of the second medical device within a portion of cancellous bone of the vertebra; and
expanding the distal end portion of the second medical device within the portion of the cancellous bone such that a cavity is formed within the cancellous bone of the vertebra.

37. The method of claim 32, wherein the biological body is an intervertebral disc, the medical device is a first medical device, the method further comprising:
after the rotating the cannula, removing the stylet from the cannula;
after the removing, inserting a second medical device through a lumen of the cannula;
positioning a distal end portion of the second medical device within a nucleus of the intervertebral disc; and
expanding the distal end portion of the second medical device within the nucleus of the intervertebral disc such that an endplate of an adjacent vertebra is moved.

38. A kit, comprising:
a cannula including a thread-forming portion on an outer wall of the cannula, the cannula being configured to form a channel having at least partially threaded interior walls in a bone; and
at least one bone screw having a threaded portion configured to matingly engage the threaded interior walls of the channel.

39. The kit of claim 38, wherein the at least one bone screw includes at least two bone screws.

40. The kit of claim 38, further comprising:
a medical device configured to be inserted at least partially through a lumen of the cannula when the cannula is inserted at least partially into a biological body, the medical device configured to perform a medical procedure within the biological body.

41. The kit of claim 38, further comprising:
an expandable device configured to be inserted through a lumen of the cannula when the cannula is inserted at least partially into a biological body, the expandable device configured to be moved from a collapsed configuration to an expanded configuration within the biological body.

42. The kit of claim 38, wherein the cannula is configured to be threadably inserted at least partially into a vertebra, the bone screw is a pedicle screw, the kit further comprising:
a fusion device configured to be secured to the vertebra with the pedicle screw.

43. The kit of claim 38, wherein a distal end of the thread-forming portion of the cannula is disposed at a non-zero distance from a distal end of the cannula.

44. The kit of claim 38, further comprising:
a stylet configured to be inserted through a lumen of the cannula and having a distal end configured to penetrate a biological body.
